(19) United States
(12) Patent Application Publication
(10) Pub. No.: US 2017/0071744 A1
(43) Pub. Date: Mar. 16, 2017

(54) Composition of orthopedic knee implant and the method for manufacture thereof

(71) Applicants: Sulzhan Bali, Tamil Nadu (IN); Lalitha Kuppuswamy, Tamil Nadu (IN)

(72) Inventors: Sulzhan Bali, Tamil Nadu (IN); Lalitha Kuppuswamy, Tamil Nadu (IN)

(21) Appl. No.: 15/265,566

(22) Filed: Sep. 14, 2016

(30) Foreign Application Priority Data
Sep. 15, 2015 (IN) 4901/CHE/2015

(51) Int. Cl. A61F 2/30; A61F 2/38

(52) U.S. Cl. CPC A61F 2/3094; A61F 2/38; A61F 2/30771; A61F 2002/3097; A61F 2002/30971; A61F 2310/00023; A61F 2310/00095; A61F 2310/00089; A61F 2002/30838

(57) ABSTRACT

The present invention discloses a composition of a knee implant comprising biomaterials such as combination of Ti—Nb—Zr alloy and tantalum to support osseointegration. The present invention further discloses a method of manufacturing customized patient-specific knee implant using 3D printing technology to suit the patient. The method involves the use of high energy source such as fiber laser or electron-beam. The base plate is mounted on the CNC. The energy source creates a melt pool on the base plate and the energy source is fed with a biomaterial in the form of wire or powder. The biomaterial is deposited on the base plate layer by layer, which solidifies in the melt pool of the base plate. The knee implant thus fabricated suits the elastic modulus of the bone and is useful as customized implant in patient undergoing replacement surgery.

### TECHNICAL FIELD OF THE INVENTION

The present invention discloses a composition of a knee implant comprising a combination of tantalum and titanium alloy, which is customized to patients undergoing orthopedic surgery or knee replacement surgery. The present invention also discloses a method of preparation for knee implants using additive manufacturing or three-dimensional (3D) printing technology. The invention further discloses a method of manufacturing patient-specific knee implants based on the 3D image of the damaged knee using 3D printing technology.

### BACKGROUND OF THE INVENTION

Osteoarthritis is a degenerative disease associated with the breakdown of joint cartilage and the underlying bone. Osteoarthritis is usually caused by aging, injury and obesity, and is associated with pain, swelling and stiffness of the joint.

Osteoarthritis is a disease generally affecting elderly humans. The risk factors thus include the genetic makeup of the individual, a lack of physical activities, excess body weight, and loss in muscle strength supporting the joint. Usually, the symptoms are not predictable unless the bone damage is observed.

Osteoarthritis is one of the leading causes of chronic disability in India and it affects over 15 million Indians each year. At present, there are no direct treatment options available for osteoarthritis. The management of osteoarthritis is broadly divided into non-pharmacological, pharmacological, and surgical treatments. The surgical treatment includes the replacement of the joint and is generally opted for in cases of failed medical management, where functional disability affects the patient's quality of life.

The knee is the largest joint in the human body and healthy knees are required to perform everyday activities. The knee is made up of the lower end of the thighbone (femur), the upper end of the shinbone (tibia), and the kneecap (patella). The point at which the ends of these three bones contact are covered with articular cartilage, a smooth substance that protects the bones and enables to locomote easily. All residual surfaces of the knee are covered by a thin lining called the synovial membrane. This membrane releases a fluid that lubricates the cartilage, reducing friction to nearly zero in a healthy knee. Any damage to the knee results in disability, which necessitates the replacement of the knee with an implant.

Knee replacement surgery was first performed in the year 1968. Since then, improvements in surgical materials and techniques have greatly increased its effectiveness. Total knee replacements are one of the most successful procedures in the field of orthopedic medicine.

Currently, surgeons in India import the readily available knee implants from USA or Europe. Usually, these implants are designed for the European and US population, and are typically available in a variety of sizes. The surgeon chooses the appropriate size of the knee implant based on the two-dimensional (2D) x-ray and measurements of the damaged knee taken on the operating table. Based on the measurements taken and the implant sizes available, the knee implant of most suitable size is chosen. In order to install the ready-made knee implant, a portion of the patient's bone is chipped off to fit the available implant. In other words, the precious irreplaceable bone of the patient is sacrificed so as to fit the readily available knee implant. This approach, in many cases, leads to a poor fitting of the knee implant, which in turn can lead to the implant incompatibility in the patient's future.

The “cut the bone to suit implant” approach and the high cost of imported knee implants have resulted in a low prevalence of knee replacement in India compared to other countries. It is also not cost effective to use the imported knee implant. Due to the high failure rate of current practices in India, the surgery is deferred until the patient's mobility becomes very poor.

The state of the art discloses the use of different types of implants as listed below.

The US Patent Application Number US 2011/0266265 A1 titled “Implant device and method for manufacture” discloses a system, device and method for optimizing the manufacture and production of patient-specific orthopedic implants. The method includes obtaining a three-dimensional image of patient's joint, selecting a standard blank implant to be optimized for the patient and finally modifying the blank implant to alter specific features of the implant to conform to the patient's anatomy. The material used to manufacture the knee implant is ceramic, metal, polymer etc. The US patent only modifies the blank by additional deposition using 3D Printing. The US Patent discloses the creation of porosity in the implant by adding a porous coating whereas in the present invention, porosity is created as an integral feature of the implant without the need of subsequent joining, welding or coating. This prevents the failure of the implant due to delamination of the coating.

The PCT Application WO 2005072785 titled “Highly porous 3 dimensional biocompatible implant structure” discloses a highly porous three-dimensional biocompatible implant structure and a process for the manufacture of the same. The porous biocompatible implant structure of the invention is virtually free of knots and welding points and exhibits a high mechanical strength. The invention provides a process for producing the biocompatible implant structure by providing one or more sheets of a ductile biocompatible material perforating the sheets to create a porosity in the range of 40-95%, coating the perforated sheets with a biocompatible material, arranging coated sheets in such a way that a three-dimensional arrangement is formed in which previously non-associated parts of the one or more perforated sheets are in direct contact; sintering the arrangement of one or more coated sheets under high vacuum. However, the invention discloses the use of perforated sheets, which results in reduced compatibility and low osseointegration of the implant. The process of assembly and/or coating creates multiple joints and leads to failures of the implant inside the human body, due to variation in corrosion properties, peel off or delamination, or the creation of gaps between coated sheets.

The U.S. Pat. No. 8,234,097 B2 titled “Automated systems for manufacturing patient-specific orthopaedic implants and instrumentation” discloses a patient-specific implant that requires minimal input from a designer or other operator and is capable of designing an implant in a small fraction of the time using computer aided design (CAD) tool. The three-dimensional implant is designed based on a medical image, such as a CT scan of a patient. This patent application primarily focuses on design, but not on metallurgical composition and processing, as well as metallurgical and biological performance.

The state of art discloses the manufacture of implants using different biomaterials and joining the biomaterials by some welding process in order to achieve better integration of porous tantalum and higher wear resistance. This makes the joint to be distinctively different in properties and may lead to premature failure due to corrosion from bio fluids or galvanic corrosion or weld zone corrosion, which accelerates the mechanical failure of the implant. In some other cases, one type of material is plasma sprayed on the other type of material. This method further leads to failure due to delamination.

Generally, Ti-6Al-4V alloy is used in majority of ready-made and 3D printed patient specific implants. The state of art shows that while titanium is biocompatible, neither aluminum nor vanadium is biocompatible. The aluminum and vanadium ions released by the implant due to wear and corrosion in the human body are toxic. Aluminum ions in blood also results in a higher risk of neurodegenerative diseases. Vanadium has been confirmed to be cytotoxic. The Ti-6Al-4V alloy is also associated with soft tissue formation. Further, the Ti-6Al-4V alloy exhibits galling wear and have a high coefficient of friction. The wear debris leads to osteolysis.

In addition to the Ti-6Al-4V alloy, a Co—Cr—Mo alloy is also widely used for the femoral component of the knee implant, since the wear resistance of Co—Cr—Mo alloy is higher than that of tantalum or titanium. However, the Co—Cr—Mo alloy is associated with poor osseointegration and poor biocompatibility. Due to its high elastic modulus, Co—Cr—Mo also results in stress shieldng of the bone. Further, cobalt ions released due to wear and corrosion is carcinogenic.

Even though, the Co—Cr—Mo alloy has a lower coefficient of friction compared to Ti-6Al-V, the high wear rate of the UHMWPE or XLPE (polyethylene) insert placed between femoral and tibial components leads to knee implant surgery failure in many cases. Therefore, there is a need to reduce the polyethylene wear. High wear of polyethylene also leads to osteolysis.

Hence, there is a need for a biocompatible knee implant that promotes osseointegration, and that possesses adequate wear resistance and low elastic modulus. There is also a need for a customized and patient-specific knee implant that fits to the damaged bone without excessive chipping (of the bone) to install the implant.

### SUMMARY OF THE INVENTION

The present invention discloses a novel composition for a knee implant comprising of biomaterials such as the combination of a Ti—Nb—Zr alloy and tantalum to promote osseointegration. The present invention further discloses a method of manufacturing the customized and patient-specific knee implant using 3D printing technology to suit the patient. The present invention also discloses the method of design and selection of the customized and patient-specific knee implant.

The present invention discloses the use of different biomaterial compositions that are patient-specific and thus depend on the particular requirements of the patient. The composition comprises of a combination of tantalum (at a percentage between 30% and 60%) and Ti—Nb—Zr alloy (for the remaining percentage) depending on the implant design. The parts or surfaces of the implant that are in contact with the bone are made of tantalum. The other parts or surfaces of the implant that are not in direct contact with the bone are made of Ti—Nb—Zr alloy. The composition is ideal for achieving optimum osseointegration and is also cost effective. The combination of these biomaterials promotes osseointegration of the knee implant with high wear resistance.

In cases where cost is a critical factor such as with Government subsidized medical treatments tantalum, which is more expensive, is not used in the composition and the implant is manufactured only with the Ti—Nb—Zr alloy.

The present invention discloses the use of additive manufacturing technology also known as 3D printing technology to manufacture accurate and patient specific knee implants. The additive manufacturing technology involves the use of a high-energy source, such as a pulsed or continuous wave fiber laser, an electron-beam, or a micro-plasma torch. The base plate is mounted on the 5-axis Computer Numerical Control (CNC) worktable in a vacuum chamber. The vacuum chamber is evacuated to ensure that the oxygen content is less than 1 ppm (parts per million) within the vacuum chamber so that the oxidation of the biomaterial is eliminated inside the vacuum chamber. The high-energy source, such as the plasma or laser beam, is mounted on the vertical Z-axis of the CNC table so that the energy source creates a melt pool on the base plate for deposition of the biomaterial. The energy source is flexible in movement within the axis and the capacity of the energy source is maintained at different powers depending on the source used. The energy source is fed with a selected biomaterial in the form of a wire (one or two sizes at a time) and/or a powder or a combination of both, which ensures economy and also good compatibility of the biomaterials. The biomaterial used in the present invention is the combination of Ti—Nb—Zr alloy and tantalum. The energy source melts the biomaterial, which is deposited on the base plate layer by layer, and which solidifies in the melt pool of the base plate. The movement of the melt pool and deposition is continued until optimum configuration of the implant is obtained by simultaneously moving the vertical energy source and the CNC table. The melting of the wire and/or the powder along with the movements of both the CNC table and the vertical energy source creates a customized, patient-specific knee implant.

In order to reduce the residual stress in the implant, it is necessary to preheat the base plate with the help of a laser or plasma torch and also to use such an external heat source during deposition in order to maintain the deposited parts at suitable temperature.

The present invention also discloses a method for the manufacture of a customized and patient-specific knee implant that overcomes the drawbacks associated with the use of readily available knee implants. An initial physical model of the damaged knee is first created from a Computed Tomography (CT) scan of the knee as the input. This physical model of the damaged knee is made from acrylonitrile butadiene styrene plastic using a 3D printer. Based on this initial plastic model, the knee implant is designed to suit damaged knee with minimal bone chipping. In the physical model of the knee implant, the parts in contact with the bone are designed to be porous so that the bone grows into the porosity to create the bond with the implant. The open pores are approximately 100 to 700 microns in size.

Once a surgeon approves the plastic physical model of the knee implant, the metal knee implant version of the approved physical plastic model is then manufactured using additive manufacturing or 3D printing technology. The dense and porous parts are deposited at the same time so as to form an integral portion of the implant. The metal knee implant is the actual knee implant, which is customized for each patient.

The knee implant is manufactured using a combination of biomaterials, such as titanium alloy and tantalum. The knee implant is patient-specific and is based on the dimensions of the damaged knee. The implant is easily fitted with minimum chipping of the bone. The use of patient-specific implant results in the accurate fixing of the implant and also reduces the risk of excessive chipping of the bone.

Usually, titanium and tantalum exhibit poor wear resistance. In order to overcome this issue, the present invention utilizes the method of in-situ hardening of the bearing surface of the femoral component of knee implant. This in-situ hardening overcomes the poor wear resistance of titanium and tantalum.

When the desired thickness of the bearing surface of femoral component of about 500 to 750 microns, is yet to be deposited, gaseous molecules of nitrogen and/or oxygen or ions of nitrogen and/or oxygen are introduced into the melt pool. This creates the formation of nitrides and/or oxides respectively. If required, a suitable negative bias voltage is also applied to the base plate, which enables the diffusion of the ion source into the melt pool. The depth and extent of the oxide and/or nitride formation is controlled by the flow rates. The objective is to obtain a functionally gradient surface such that the softest portion is the interior bearing surface, and that the hardest portion is the exterior bearing surface. As a result, titanium nitride (TiN), tantalum nitride (TaN), titanium dioxide (TiO₂) or tantalum pentoxide (Ta₂O₅) is formed depending on the source selected, and are biocompatible.

Another method is to introduce nanopowders of TiO₂, TiN, TiC, Ta₂O₅ or TaN into the melt pool during the oxidation and/or nitriding steps.

The depth of hardening should include machining allowance, so that the depth of the hardened zone in the finished component is at least 0.3 mm (300 microns).

The hardness of the oxide/nitride layer is greater than that of the Co—Cr—Mo alloy presently used. This process also reduces the coefficient of friction between the femoral component and the UHMWPE or XLPE insert. In addition, due to the better wettability of these layers, contact with the body fluids is improved resulting in superior lubrication. Therefore, the wear of the insert, as well as the wear of the femoral component, are substantially reduced.

### BRIEF DESCRIPTION OF THE DRAWINGS

The features of the embodiments will become clearer from the detailed descriptions that follow, particularly when read together with the accompanying figures.

FIG. 1 illustrates a flow chart depicting the manufacturing method for the knee implant.

FIG. 2 illustrates a flow chart illustrating the design and selection of the customized and patient-specific knee implant.

### DETAILED DESCRIPTION OF THE INVENTION

In order to clearly and concisely describe the subject matter of the claimed invention, we provide definitions for specific terms that are subsequently used in the written description.

The term “Implant” refers to a substitute for the damaged bone, tissue etc., which is fabricated artificially to match the damaged portion of the bone, and which is attached to a patient undergoing knee replacement surgery.

The term “Alloy” refers to a metal made by combining two or more metallic elements, particularly to provide greater strength or resistance to corrosion.

The present invention discloses a novel composition of a knee implant biomaterials such as the combination of a Ti—Nb—Zr alloy and tantalum to promote osseointegration. The present invention further discloses a method of manufacturing a customized and patient-specific knee implant using 3D printing technology.

The biomaterials are combined in different proportions depending on the requirements of the patient (i.e., the biomaterial combinations are patient-specific). The first composition of the biomaterials consists of 100% tantalum, since tantalum exhibits an improved rate of osseointegration. The second composition consists of 100% Ti—Nb—Zr alloys, such as Ti-10Nb-10Zr, Ti-13Nb-13Zr and Ti-23Nb-13Zr, which are also preferred for improved osseointegration. The third composition consists of combinations of tantalum (between 30% to 60%) and Ti—Nb—Zr alloy (between 40% to 70%) depending on the implant design. In this type of implant, the parts or surfaces of the implant that are in contact with the bone are made of tantalum. The other parts of the implant, which are not in direct contact with the bone, are made of Ti—Nb—Zr alloy. This combination of tantalum and Ti—Nb—Zr alloy represents the ideal implant composition to achieve optimum osseointegration, and it is also cost effective.

The knee implant is manufactured using combination of biomaterials such as Ti—Nb—Zr alloy and tantalum. The combination of these biomaterials promotes osseointegration of the knee implant with high wear resistance. Tantalum is used in areas of the knee implant that are in contact with the bone due to its better osseointegration, as compared to the Ti—Nb—Zr alloy. At the same time, the surface of the implant, where there is no bone contact, is made of Ti—Nb—Zr alloy since it is less expensive compared to tantalum. The use of wire and powdered forms of the biomaterials facilitate specific biomaterial configurations for the patient-specific knee implant, in a cost effective manner.

The present invention also discloses the method of manufacturing the customized and patient-specific knee implant. The present invention further overcomes the problems associated with the use of readily available knee implants, through patient-specific knee implants.

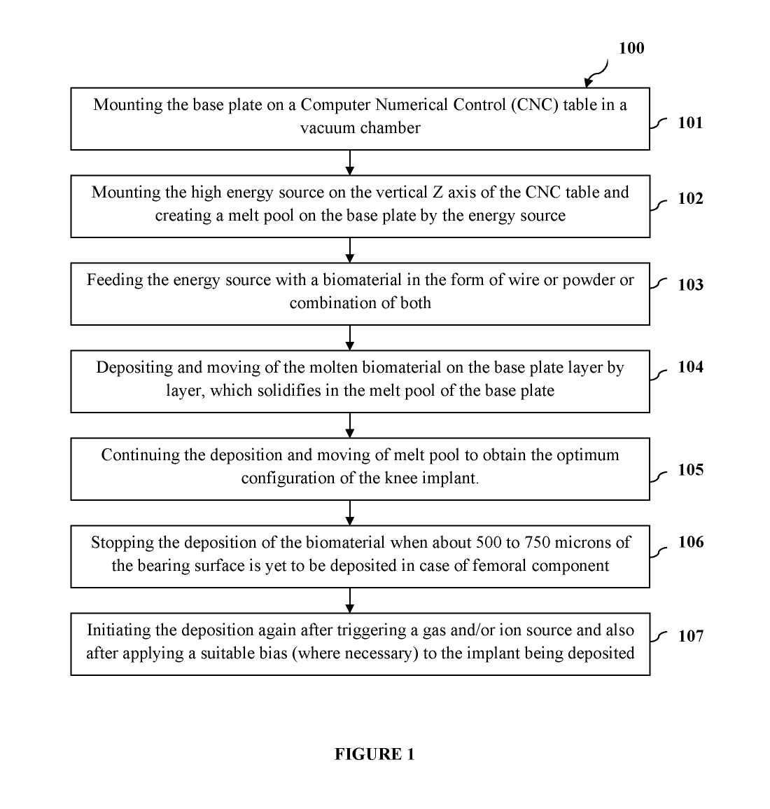

FIG. 1 illustrates a flow chart depicting the manufacturing method for the knee implant. The present invention discloses the use of additive manufacturing technology, also termed as 3D printing technology, to manufacture accurate and patient-specific knee implants. The additive manufacturing technology involves the use of a high-energy source such as a fiber, pulsed laser, electron-beam, or micro-plasma torch. Further, the method involves the use of an inexpensive wire made of a suitable biomaterial. The method 100 of using additive manufacturing technology for the manufacture of a knee implant begins at step 101, where a base plate is mounted on a Computer Numerical Control (CNC) table in a vacuum chamber. The vacuum chamber is evacuated to ensure that the oxygen content is less than 1 ppm within the vacuum chamber, so that oxidation of the biomaterial is eliminated inside the vacuum chamber. At step 102, the high-energy source such as a plasma torch or laser beam is mounted on the vertical Z-axis of the CNC table such that the energy source creates a melt pool on the base plate for the deposition of the biomaterial. The energy source is flexible in movement within the axis and the capacity of the energy source is maintained at 1 to 2 kW (Kilo Watt) for the plasma torch, or between 200 to 500 W for continuous wave (cw) fiber laser, or a 20 kW peak in the case of a pulsed laser. At step 103, the energy source is fed with a suitable biomaterial in the form of a wire or powder or a combination of both. In addition, the process allows for a change in biomaterial used depending on the specific requirement. The biomaterial used in the present invention is a combination of a Ti—Nb—Zr alloy and tantalum. At step 104, the biomaterial is melted by the energy source and is deposited on the base plate, layer by layer. The deposited biomaterial solidifies in the melt pool of the base plate. At step 105, the movement of the melt pool and deposition is continued in order to obtain the optimum configuration of the implant. The rate of deposition of the biomaterial usually varies between 50 gms per hour and 500 gms per hour. The implant is created as per the required configuration through the simultaneous movement of the vertical energy source and the CNC table. The process parameters such as laser beam and/or plasma torch power, feed rate of wire and/or powder, speed of the CNC system and pitch or distance between deposition tracks are varied accordingly such that the porosity the deposition is varied from 1% to 80% as required. The porous parts of the implants are integral parts of the implant and are deposited at the same time and they are not welded or coated. The porosity is very low, i.e., 1% to 5% in the bearing surface of the femoral component and can be as high as 80% in the parts in contact with the bone so as to allow the bone to grow into the pores. The size of the pores is typically 100 to 700 microns. The elastic modulus of the implant is matched with that of the bone by varying the porosity and/or biomaterial or both. The melting of the wire and/or the powder, along with the movements of the CNC table and vertical energy source, creates a customized, patient-specific knee implant. At step 106, in the case of the femoral component, the deposition of the biomaterial is stopped when about 500 to 750 microns of the bearing surface is yet to be deposited. At step 107, the deposition is initiated again, after triggering the flow of an ion and/or gas source such as oxygen or nitrogen, and also after applying a suitable bias to the implant being deposited (where necessary). The process results in the fabrication of one implant in 1 to 4 hours depending on the feed rate, the number of biomaterials used, etc.

The porosity of the implant plays an important role in promoting the growth of the bone, and for osseointegration. The porosity of the knee implant is altered by varying the deposition current of the energy source, the feed rate of the wire or powder and also by changing the pitch or distance between the tracks. The process further allows the porosity of the implant, composed of Ti—Nb—Zr alloy and tantalum, to be altered so that the elastic modulus of the implant matches that of the bone.

Where it is in contact with the bone, the implant's porosity promotes osseointegration by enabling the bone to grow into the pores. In order to achieve load transfer from the implant to the adjoining bone, the elastic moduli have to match. Porosity helps reduce the elastic modulus of the implant so as to match that of the bone. This helps avoiding of stress shielding and also enables load transfer to the bone, ensuring a healthy bone.

The use of inorganic compounds through the space-holder-technique can also alter the porosity of the knee implant. Sodium chloride or ceramic oxide is added to the melt pool of the base plate, which does not mix with the metal or alloy. The process also enables substantial variation of porosity. Low porosity in the range of 1% to 5% is maintained on the surface of the implant, where wear resistance is required. In contrast, high porosity of 75% to 80% is maintained on the inner surface of the implant, which is in contact with the bone. Osseointegration, or cement-less bonding of the bone to the implant, requires the bone to grow into the pores of the implant, thereby creating the bond. Hence, for the bone to grow into the pores of the implant, pore sizes must be in a specific range—typically between 100 to 700 microns—which is achieved by the present invention. The present invention enables the creation of porosity as an integral feature of the implant, without resorting to subsequent joining, welding or coating.

Since the knee implant is manufactured by the simultaneous deposition of biomaterials, it exhibits functionally gradient properties without the risk of failure.

The method disclosed in the present invention produces a knee implant surface that is continuous and dense. The implant surface acts as a bearing surface, i.e., one where there is movement during the regular functioning of the knee. At the same time, the surface in contact with the bone is porous so as to promote osseointegration and to help match the elastic modulus of the bone. In other words, the same material can be deposited as dense with very low porosity, or as highly porous, by changing the deposition conditions. This minimizes stress shielding of the bone, and improves the reliability of the joint replacement surgery.

Titanium and tantalum exhibit poor wear resistance. In order to overcome this limitation, the present invention utilizes a method of in-situ hardening of the bearing surface of the knee implant. During deposition of the biomaterial, the bearing surface of the femoral component of the knee implant is subjected to hardening.

Hardening the surface of the femoral component is achieved in one of the following ways:

The first method includes nitriding or oxidation, which is done in-situ during the deposition of the last few layers, usually for a thickness of 0.5 to 0.75 mm (500 to 750 microns). During the deposition, oxygen and/or nitrogen is introduced into the melt pool, along laser beam or a plasma torch, which results in the formation of oxides or nitrides. The extent of oxide or nitride formation is controlled by the flow rate of oxygen and/or nitrogen. It may be necessary to apply a negative bias on the work piece to improve the hardening process. The objective is to obtain a functionally gradient surface, such that the softest portion is in contact with the interior of the implant and the hardest portion is on the exterior portion of the implant. The depth of hardening should include a machining allowance, so that the depth of hardening is at least 0.3 mm in the final implant. Further, it may also be necessary to introduce oxygen and/or nitrogen as ions from an ion source rather than only as gas molecules.

Another method is to introduce nanopowders of titanium dioxide (TiO₂), titanium nitride (TiN), titanium carbide (TiC), tantalum pentoxide (Ta₂O₅), or tantalum nitride (TaN) into the melt pool during the oxidation and/or nitriding steps. The titanium nitride (or tantalum nitride), titanium carbide (or tantalum carbide), and titanium dioxide (or tantalum pentoxide) thus formed are biocompatible.

The hardness (including scratch hardness) of the in-situ hardened layers of the knee implant is higher than that of the Co—Cr—Mo implant, which is presently used. Moreover the co-efficient of the friction is less than that of the Co—Cr—Mo implant. These in-situ hardened layers of the implant also exhibit better wettability of the body fluids compared to the traditional Co—Cr—Mo implant. These factors result in a higher wear resistance of the femoral component, but at the same time they result in a reduction in the wear of the polyethylene insert. This method of in-situ hardening improves the overall wear resistance of the knee implant.

This method of in-situ hardening improves the wear resistance of tantalum and titanium alloys and improves the surface hardness of the femoral component to reduce wear during its movement. This surface hardening is being done in-situ during deposition and not as an additional step, to achieve the requisite hardening depth and to minimize the steps involved in the manufacture of the knee implant.

The porous parts of the implant are electrochemically oxidized so as to form multiple oxide nanotubes on the surface. The nanoporous structure on the porous surface of the implant promotes osseointegration after the implant is installed in the patient. Prior to electrochemical oxidation, it is preferable to laser texture the surface of the porous parts of the implant by traversing or scanning the laser beam on the surface of the implant. Both the in-situ hardened parts and the porous parts are produced in the same manufacturing process, and therefore represent integral parts of the implant. There are no welding or coating requirements.

Regulatory authorities require consistency, repeatability and reliability in the implant manufacturing process. As a result, process control is critical. The 3D printing process disclosed in the present invention is associated with a continuous monitoring of the melt pool temperature and dimensions and also bead dimensions through three cameras, one 2-wavelength pyrometer, and suitable image processing algorithms. These enable the generation of a microstructure map between process conditions and implant microstructure (and hence the metallurgical properties) at a given location.

The elastic modulus of the Ti—Nb—Zr alloy chosen is substantially less than the Ti-6Al-V alloy. The elastic modulus is further reduced through the porous structure, so as to match the elastic modulus of the bone. This ensures load transfer from the implant to the bone, improving the density and life of the bone. In other words, the present invention overcomes the stress shielding limitation exhibited by current implants.

Usually, the 3D printing process does not offer surfaces with high surface finish. The present invention minimizes this problem by using wire in portions or areas that require high surface finish. The use of wire offers a better finish compared to using powder. However, final machining and polishing are still necessary to achieve the requisite dimensional tolerance and surface finish.

Laser machining with nano and picosecond lasers to 30-micron accuracy is possible. However, it is not feasible to use this as a finishing step immediately after 3D printing, due to possible dimensional variation from the annealing step. Therefore, to avoid the high cost of conventional 5-axis CNC finish milling, laser machining is performed in the present invention after annealing in the same 3D printing setup to achieve the requisite final dimensions and surface finish.

The surface structure of the porous part of the implant plays an important role in the osseointegration. In order to improve the rate of osseointegration, the surface of the implant, which is in contact with the bone, is electrochemically oxidized to create multiple oxide nanotubes on the surface inducing porosity. The nanoporous structure on the surface of the implant promotes improved osseointegration allowing the growth of the osteoblasts after the implant is installed in the patient.

In order to improve the osseointegration further, the porous parts are laser textured by traversing or scanning a laser beam, prior to electrochemical oxidation.

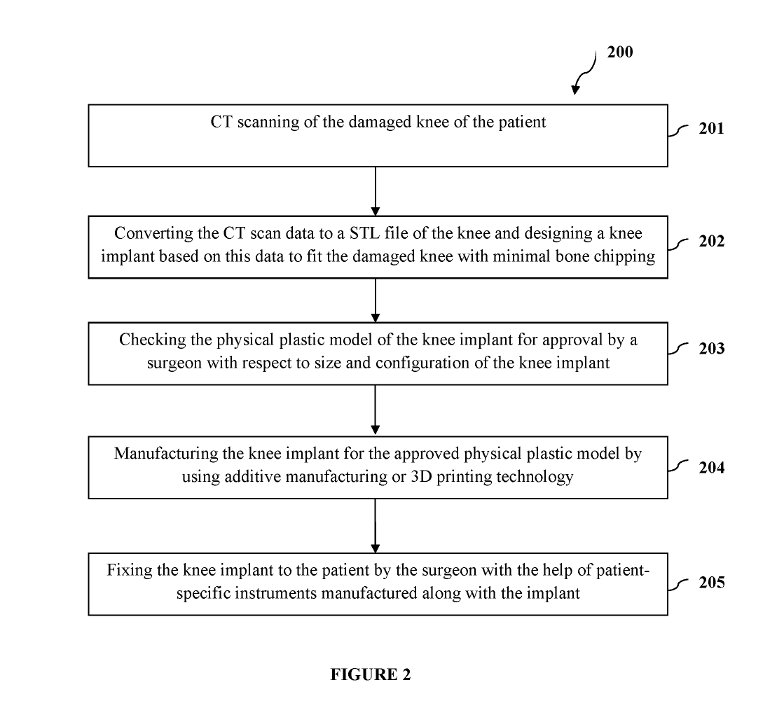

FIG. 2 illustrates a flow chart illustrating the design and selection of the customized and patient-specific knee implant. The method 200 starts with step 201 of Computer Tomography (CT) scanning of the damaged knee of the patient. At step 202, the CT scan data is converted to a Standard Template Library (STL) file of the knee. A physical model of the knee implant is designed to fit the damaged knee in a patient. The physical model of the knee implant is prepared using acrylonitrile butadiene styrene plastic or any suitable polymer. The physical plastic model is patient-specific and is produced in order to check the accuracy of the size and configuration of the knee implant. The knee implant is designed to fit the damaged knee with minimal bone chipping. At step 203, the physical plastic model of the knee implant is checked for approval by the surgeon with respect to size and configuration of the knee implant. At step 204, the metal knee implant is manufactured as per the approved physical plastic model by using additive manufacturing technology or 3D printing technology. The metal knee implant is the actual knee implant, which is customized to each patient. The knee implant is manufactured using the combination of biomaterials such as titanium alloy and tantalum using additive manufacture technology. At step 205, the surgeon fixes the knee implant to the patient with the help of patient-specific instruments manufactured along with the implant. As the knee implant is patient specific, and is based on the dimensions of the damaged knee, installation is convenient for the surgeon with minimum chipping of the bone in order to fix the knee implant.

The present invention discloses the use of wire and powder forms of the biomaterial to obtain the biocompatible knee implant. The present invention also discloses the creation of porosity as an integral feature of the implant.

The present invention further discloses the in-situ hardening of the bearing surface as an integral part of the process.

The biomaterials used in the present invention such as Ti—Nb—Zr alloy and tantalum are superior in terms of biocompatibility and osseointegration, compared to Cobalt-Chromium (Co—Cr) alloy usually used in current implants. The Ti—Nb—Zr alloy comprises titanium, niobium and zirconium, all of which are biocompatible and do not result in toxicity or incompatibility in the patient.

The process disclosed in the present invention is also useful for the manufacture of suitable patient-specific jigs, fixtures, and gauges that aid the surgeon during surgery using 3D printing technology. These jigs, fixtures, and gauges may be produced in any suitable biocompatible and sterilizable polymer. This ensures better fit between the bone and implant.

The knee implant produced by the present invention matches the elastic modulus of the bone, which is achieved by using a low modulus Ti alloy and by creating porosity in the implant.

Another advantage of the present invention is the use of the Android operating system, which enables easy communication and transfer of models and images to the surgeon through smart phones and tablets.

The present invention is not only specific or restricted to knee implants. The similar composition and the method of preparation are also applicable for other implants such as the hip, etc.

We claim:

1. A composition of a knee implant for a patient undergoing replacement surgery, the knee implant comprising:
a. tantalum at a concentration of 30% to 60%; and
b. a Ti—Nb—Zr alloy at the concentration of 40% to 70%.

2. The composition as claimed in claim 1, wherein the surface of the implant in contact with a bone is made of tantalum and the surface of the implant not in contact with the bone is made of Ti—Nb—Zr alloy.

3. A method for manufacture of a knee implant, wherein the method uses an additive manufacturing technology or 3-Dimentional (3D) printing technology, the method comprises the steps of:
a. mounting a base plate on a Computer Numerical Control (CNC) table in a vacuum chamber;
b. mounting a high energy source such as a micro-plasma torch or a laser beam on a vertical Z axis of the CNC table such that the energy source creates a melt pool on the base plate;
c. feeding the energy source with a biomaterial in the form of a wire or a powder or a combination of both;
d. allowing the biomaterial to melt from the energy source and depositing the melted biomaterial on the base plate layer by layer and allowing the layers to solidify in the melt pool of the base plate;
e. continuing the movement of melt pool and deposition by simultaneously moving the vertical energy source and the CNC table to obtain an optimum configuration of the knee implant;
f. stopping the deposition of the biomaterial when about 500 to 750 microns of the bearing surface is yet to be deposited in case of femoral component; and
g. in-situ surface hardening by initiating the deposition again after triggering the flow of a gas or ion source such as oxygen and/or nitrogen into the melt pool and also after applying a suitable bias, where necessary, to an implant being deposited.

4. The method as claimed in claim 3, wherein the vacuum chamber is evacuated to ensure that the oxygen content is less than 1 ppm (parts per million) so as to eliminate oxidation of the biomaterial.

5. The method as claimed in claim 3, wherein the energy source is flexible in movement within the axis and the capacity of the energy source is maintained at 1 to 2 kW (Kilo Watt) for plasma torch or 500 W for cw fiber laser or 20 kW peak for pulsed laser.

6. The method as claimed in claim 3, wherein in-situ surface hardening of the implant is achieved by introducing nanopowder selected from a group comprising titanium dioxide (TiO2), titanium nitride (TiN), titanium carbide (TiC), tantalum pentoxide (Ta2O5), or tantalum nitride (TaN) into the melt pool.

7. The method as claimed in claim 3, wherein the biomaterial is a combination of Ti—Nb—Zr alloy and tantalum.

8. The method as claimed in claim 3, wherein the rate of deposition of the biomaterial varies between 50 gms per hour to 500 gms per hour.

9. The method as claimed in claim 3, wherein the surface of the implant in contact with a bone is electrochemically oxidized to create multiple oxide nanotubes such that increasing the porosity and promoting osseointegration by traversing or scanning of laser beam on one or more porous parts of the implant prior to electrochemical oxidation.

10. The method as claimed in claim 3, wherein the porosity of the implant is altered through space-holder-technique by adding sodium chloride or ceramic oxide to the melt pool of the base plate such that porosity of the implant achieved is 100 to 700 microns.

11. The method as claimed in claim 3, wherein the elastic modulus of the implant is matched with that of the bone by varying the porosity of the implant and the biomaterial.

12. A method for selection of customized and patient-specific knee implant, the method comprises the steps of:
a. subjecting a damaged knee of a patient to a Computer Tomography (CT) scanning;
b. converting the CT scan data to a Standard Template Library (STL) file of the knee and designing a knee implant based on this data to fit the damaged knee with minimal bone chipping;
c. checking the physical model of the knee implant for approval by a surgeon with respect to size and configuration of the knee implant;
d. manufacturing a metal knee implant for the approved physical model by using additive manufacturing technology; and
e. fixing the metal knee implant into the patient along with one or more patient-specific instruments manufactured along with the implant.

13. The method as claimed in claim 12, wherein the physical model of the knee implant is prepared using acrylonitrile butadiene styrene plastic.

14. The method as claimed in claim 12, wherein the knee implant is patient specific resulting in accurate fixation with minimal bone chipping.
